(19) United States
(12) Patent Application Publication
(10) Pub. No.: US 2025/0082307 A1
(43) Pub. Date: Mar. 13, 2025

(54) ULTRASONIC DIAGNOSTIC APPARATUS

(71) Applicant: Canon Medical Systems Corporation, Otawara-shi (JP)

(72) Inventors: Akio GODA, Otawara (JP); Ryosuke IWASAKI, Otawara (JP)

(73) Assignee: Canon Medical Systems Corporation, Otawara-shi (JP)

(21) Appl. No.: 18/827,132

(22) Filed: Sep. 6, 2024

(30) Foreign Application Priority Data
Sep. 7, 2023 (JP) 2023-145670
Sep. 5, 2024 (JP) 2024-153443

(51) Int. Cl. A61B 8/00; A61B 8/06; A61B 8/08; G01S 7/52

(52) U.S. Cl. CPC A61B 8/54; A61B 8/481; G01S 7/5202; A61B 8/06; A61B 8/488

(57) ABSTRACT

According to one embodiment, an ultrasonic diagnostic apparatus includes an ultrasonic probe and processing circuitry. The ultrasonic probe has a plurality of elements and is configured to transmit an ultrasonic beam a plurality of times. The plurality of elements is divided in an elevation direction. The processing circuitry is configured to control a transmission aperture of the ultrasonic probe so as to match an area and a position of elements which form one of ultrasonic beams transmitted the plurality of times with an area and a position of elements which form remaining ultrasonic beams.

### CROSS-REFERENCE TO RELATED APPLICATIONS

This application is based upon and claims the benefit of priority from Japanese Patent Application No. 2023-145670, filed Sep. 7, 2023, and Japanese Patent Application No. 2024-153443, filed Sep. 5, 2024, the entire contents all of which are incorporated herein by reference.

### FIELD

Embodiments described herein relate generally to an ultrasonic diagnostic apparatus.

### BACKGROUND

Contrast-enhanced imaging is an imaging method using an ultrasonic diagnostic apparatus. Contrast-enhanced imaging is an imaging method of receiving, a plurality of times, an ultrasonic wave having undergone phase modulation or amplitude modulation upon administration of an ultrasonic contrast medium such as microbubbles and imaging a harmonic signal as a nonlinear component. In this case, contrast-enhanced imaging is roughly classified into a phase modulation method (PM method) and amplitude modulation method (AM method). The PM method is a technique applied to B-mode tissue harmonic imaging (THI) in which the sound pressure is dropped to a level at which a contrast medium easily resonates. Accordingly, this technique is good in spatial resolution but poor in tissue bubble contrast ratio and depth sensitivity. In contrast to this, the AM method is a technique with improved tissue cancellation performance. This technique is lower in spatial resolution than the PM method but is higher in tissue bubble contrast ratio and depth sensitivity than the PM method. Typical schemes of the AM method are a voltage control scheme and a transmission aperture control scheme. The voltage control scheme has its limit on the voltage control accuracy of a pulser, and hence the transmission aperture control scheme is generally used.

Mainly used transmission aperture control schemes are a transmission aperture division scheme and a sparse transmission aperture scheme. The transmission aperture division scheme is a scheme which divides transmission apertures like setting all the elements as transmission apertures or setting half of the elements as transmission apertures. The sparse transmission aperture scheme is a scheme which controls transmission apertures by selectively using even- and odd-numbered channels. The transmission aperture division scheme varies in cancellation performance at end portion sides and a central portion of an image. In addition, since the transmission apertures are small, a bioacoustic shadow affects the image. In contrast to this, the sparse transmission aperture scheme has a problem that crosstalk from transmitting channels to non-transmitting channels has large influence.

### BRIEF DESCRIPTION OF THE DRAWINGS

FIG. 1 is a block diagram showing an example of the arrangement of an ultrasonic diagnostic apparatus according to the first embodiment.

FIG. 2 is a conceptual diagram showing the definition of an ultrasonic beam direction according to the first embodiment.

FIG. 3 is a view showing the first example of a transmission aperture control scheme according to the first embodiment.

FIG. 4 is a view showing the second example of the transmission aperture control scheme according to the first embodiment.

FIG. 5 is a view showing the third example of the transmission aperture control scheme according to the first embodiment.

FIG. 6 is a view showing the fourth example of the transmission aperture control scheme according to the first embodiment.

FIG. 7 is a view showing the fifth example of the transmission aperture control scheme according to the first embodiment.

FIG. 8 is a view showing the sixth example of the transmission aperture control scheme according to the first embodiment.

FIG. 9 is a flowchart showing contrast-enhanced imaging processing according to the first embodiment.

FIG. 10 is schematic views showing a contrast-enhanced image collected by the transmission aperture control scheme according to the first embodiment.

FIG. 11 is a flowchart showing switching processing in the transmission aperture control scheme according to the first embodiment.

FIG. 12 is a view showing the first example of a transmission aperture control scheme according to the second embodiment.

FIG. 13 is a view showing the second example of the transmission aperture control scheme according to the second embodiment.

FIG. 14 is a view showing the third example of the transmission aperture control scheme according to the second embodiment.

FIG. 15 is a view showing the fourth example of the transmission aperture control scheme according to the second embodiment.

### DETAILED DESCRIPTION

In general, according to one embodiment, an ultrasonic diagnostic apparatus includes an ultrasonic probe and processing circuitry. The ultrasonic probe has a plurality of elements and is configured to transmit an ultrasonic beam a plurality of times, the plurality of elements is divided in an elevation direction. The processing circuitry is configured to control a transmission aperture of the ultrasonic probe so as to match an area and a position of elements which form one of ultrasonic beams transmitted the plurality of times with an area and a position of elements which form remaining ultrasonic beams.

An ultrasonic diagnostic apparatus according to this embodiment will be described below with reference to the accompanying drawings. In the following embodiments, portions denoted by the same reference numerals perform the same operations, and a redundant description will be appropriately omitted.

### First Embodiment

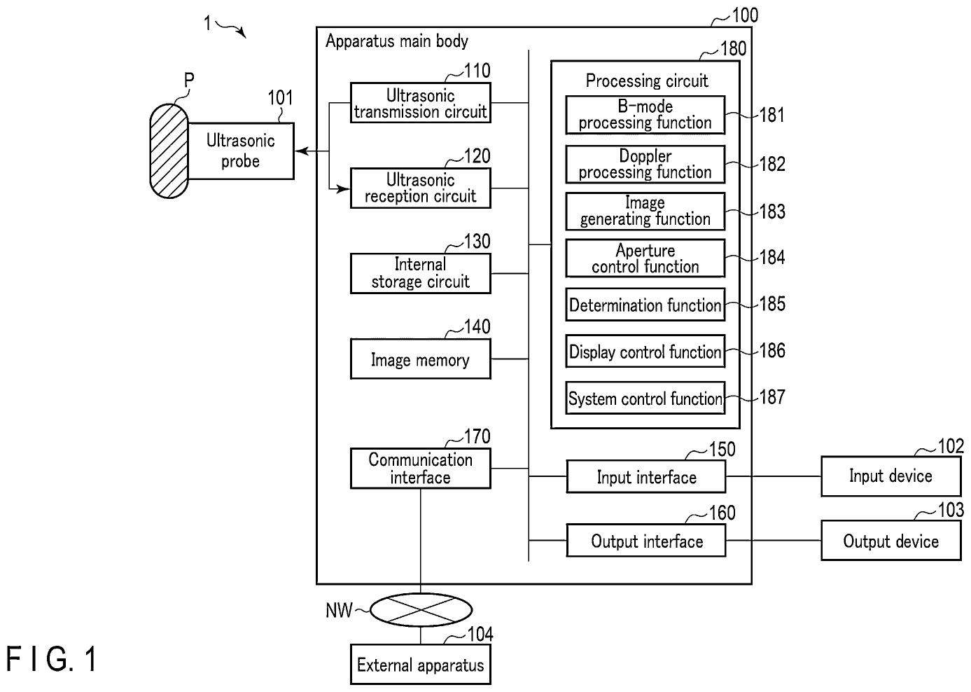

FIG. 1 is a view showing an example of the arrangement of an ultrasonic diagnostic apparatus according to this embodiment. An ultrasonic diagnostic apparatus 1 in FIG. 1 includes an apparatus main body 100 including a probe controller and an ultrasonic probe 101. The apparatus main body 100 is connected to an input device 102 and an output device 103. The apparatus main body 100 is also connected to an external apparatus 104 via a network NW. The external apparatus 104 is, for example, a server equipped with PACS (Picture Archiving and Communication Systems) or a workstation that can execute post processing.

The ultrasonic probe 101 executes, for example, ultrasonic scanning on a scan region in a living body P as an object under the control of the apparatus main body 100. The ultrasonic probe 101 includes, for example, an acoustic lens, one or more matching layers, a plurality of transducers (piezoelectric elements), and a backing member. The acoustic lens is formed from, for example, silicone rubber and focuses an ultrasonic beam. The one or more matching layers perform impedance matching between a plurality of transducers and the living body. The backing member prevents ultrasonic waves from propagating backward from the plurality of transducers in the radiation direction. The ultrasonic probe 101 is assumed to be, for example, a linear probe or a convex probe but may be a radial probe that can perform 360° scanning. The ultrasonic probe 101 is detachably connected to the apparatus main body 100. The ultrasonic probe 101 may be provided with buttons that are pressed to perform offset processing and an operation of freezing an ultrasonic image (freezing operation).

A plurality of transducers generate ultrasonic waves based on a driving signal supplied from an ultrasonic transmission circuit 110 (to be described later) of the apparatus main body 100. With this operation, the ultrasonic probe 101 transmits the ultrasonic waves to the living body P. When the ultrasonic probe 101 transmits the ultrasonic waves to the living body P, the transmitted ultrasonic waves are sequentially reflected by an acoustic-impedance discontinuous surface in the body tissue of the living body P, and a plurality of piezoelectric transducers receive the reflected waves as an echo signal. The amplitude of the received echo signal depends on acoustic impedance differences on the discontinuous surface by which the ultrasonic waves are reflected. The echo signal produced when a transmitted ultrasonic pulse is reflected by a moving blood flow is subjected to a frequency shift depending on the velocity component of the moving body in the ultrasonic transmission direction due to the Doppler effect. The ultrasonic probe 101 receives the echo signal from the living body P and converts it into an electrical signal.

FIG. 1 exemplarily shows the connection relationship between one ultrasonic probe 101 and the apparatus main body 100. However, a plurality of ultrasonic probes can be connected to the apparatus main body 100. One of the plurality of connected ultrasonic probes 101 can be arbitrarily selected to be used for ultrasonic scanning by operating a software button on a touch panel (to be described later).

The apparatus main body 100 is an apparatus that generates an ultrasonic image based on an echo signal received by the ultrasonic probe 101. The apparatus main body 100 includes an ultrasonic transmission circuit 110, an ultrasonic reception circuit 120, an internal storage circuit 130, an image memory 140, an input interface 150, an output interface 160, a communication interface 170, and a processing circuit 180.

The ultrasonic transmission circuit 110 is a processor that supplies a driving signal to the ultrasonic probe 101. The ultrasonic transmission circuit 110 is implemented by, for example, a trigger generating circuit, a delay circuit, and a pulser circuit. The trigger generating circuit repeatedly generates rate pulses for the formation of transmission ultrasonic waves at a predetermined rate frequency. The delay circuit gives each rate pulse generated by the trigger generating circuit a delay time for each of a plurality of piezoelectric transducers which is necessary to focus an ultrasonic wave generated from the ultrasonic probe into a beam and determine transmission directivity. The pulser circuit applies a driving signal (driving pulse) to the plurality of ultrasonic transducers provided in the ultrasonic probe 101 at the timing based on the rate pulse. Changing the delay time given to each rate pulse using the delay circuit can arbitrarily adjust the transmission directions from the surfaces of a plurality of piezoelectric transducers.

The ultrasonic transmission circuit 110 can arbitrarily change the output intensity of ultrasonic waves by using a driving signal. The ultrasonic diagnostic apparatus can reduce the influence of the attenuation of ultrasonic waves in the living body P by increasing the output intensity. The ultrasonic diagnostic apparatus can acquire an echo signal with a high signal-to-noise ratio (SNR) at the time of reception by reducing the influence of the attenuation of ultrasonic waves.

In general, as ultrasonic waves propagate in the living body P, the intensity of vibrations (to be also referred to as acoustic power) of ultrasonic waves corresponding to the output intensity attenuates. The attenuation of acoustic power is caused by absorption, scattering, reflection, and the like. In addition, the degree of attenuation of acoustic power depends on the frequency of ultrasonic waves and the distances of ultrasonic waves in the radiation directions. For example, increasing the frequency of ultrasonic waves will increase the degree of attenuation. In addition, increasing the distances of ultrasonic waves in the radiation directions will increase the degree of attenuation.

The ultrasonic reception circuit 120 is a processor that generates a reception signal by performing various types of processing for an echo signal received by the ultrasonic probe 101. The ultrasonic reception circuit 120 generates a reception signal with respect to the echo signal of ultrasonic waves acquired by the ultrasonic probe 101. More specifically, the ultrasonic reception circuit 120 is implemented by, for example, a preamplifier, an A/D converter, a demodulator, and a beamformer (adder). The preamplifier performs gain correction processing by amplifying the echo signals received by the ultrasonic probe 101 for each channel. The A/D converter converts a gain-corrected echo signal into a digital signal. The demodulator demodulates the digital signal. The beamformer gives, for example, each demodulated digital signal a delay time required to determine reception directivity and adds a plurality of digital signals given with delay times. The beamformer generates a reception signal with an emphasized reflection component from a direction corresponding to the reception directivity by addition processing. Note that a reception signal may be called an IQ signal. In addition, the ultrasonic reception circuit 120 may cause the internal storage circuit 130 (to be described later) to store a reception signal or may output the signal to the external apparatus 104 via the communication interface 170.

The internal storage circuit 130 has, for example, a storage medium that can be read by a processor, such as a magnetic storage medium, an optical storage medium, or a semiconductor memory. The internal storage circuit 130 stores a program for implementing ultrasonic transmission/reception, various types of data, and the like. The program and various types of data may be stored in advance in, for example, the internal storage circuit 130. Alternatively, the program and various types of data may be stored in, for example, a non-transient storage media and distributed and may be read out from the non-transient storage media and installed in the internal storage circuit 130. In addition, the internal storage circuit 130 stores B-mode image data, contrast-enhanced image data, image data concerning a blood flow video, and the like which are generated by the processing circuit 180 in accordance with operations input via the input interface 150. The internal storage circuit 130 can also transfer stored image data to the external apparatus 104 or the like via the communication interface 170. Note that the internal storage circuit 130 may store the reception signal (IQ signal) generated by the ultrasonic reception circuit 120 or transfer the signal to the external apparatus 104 or the like via the communication interface 170.

Note that the internal storage circuit 130 may be a drive device or the like that reads and writes various types of information with respect to portable storage media such as a CD drive, a DVD drive, and a flash memory. The internal storage circuit 130 can write stored data in a portable storage medium and cause the external apparatus 104 to store the data via the portable storage medium.

The image memory 140 has, for example, a storage medium that can be read by a processor, such as a magnetic storage medium, an optical storage medium, or a semiconductor memory. The image memory 140 saves image data corresponding to a plurality of frames immediately before a freeze operation input via the input interface 150. Image data stored in the image memory 140 is, for example, continuously displayed (cine-displayed).

The internal storage circuit 130 and the image memory 140 described above need not always be implemented by independent storage devices. The internal storage circuit 130 and the image memory 140 may be implemented by a single storage device. Alternatively, the internal storage circuit 130 and the image memory 140 each may be implemented by a plurality of storage devices.

The input interface 150 accepts various types of instructions from the operator via the input device 102. The input device 102 includes, for example, a mouse, a keyboard, panel switches, slider switches, a trackball, a rotary encoder, an operation panel, and a TCS (Touch Command Screen). The input interface 150 is connected to the processing circuit 180 via, for example, a bus, converts an operation instruction input from the operator with respect to the input device 102 into an electrical signal, and outputs the electrical signal to the processing circuit 180. Note that the input interface 150 is not limited to only those that are connected to physical operation components such as a mouse and a keyboard. Examples of the input interface include a circuit that accepts an electrical signal corresponding to an operation instruction input from an external input device provided independently of the medical information processing apparatus 1 and outputs the electrical signal to the processing circuit 180.

The output interface 160 is an interface for outputting an electrical signal from the processing circuit 180 to an output device 103. The output device 103 is an arbitrary display such as a liquid crystal display, an organic EL display, an LED display, a plasma display, or a CRT display. The output device 103 may be a touch panel display also serving as the input device 102. The output device 103 may further include a loudspeaker that outputs sound in addition to the display. The output interface 160 is connected to the processing circuit 180 via, for example, a bus and outputs an electrical signal from the processing circuit 180 to the output device 103.

The communication interface 170 is connected to the external apparatus 104 via, for example, the network NW and performs data communication with the external apparatus 104.

The processing circuit 180 is a processor functioning as the main unit of the ultrasonic diagnostic apparatus 1. The processing circuit 180 executes a program stored in the internal storage circuit 130 and implements a function corresponding to the program. The processing circuit 180 includes, for example, a B-mode processing function 181, a Doppler processing function 182, an image generating function 183, an aperture control function 184, a determination function 185, a display control function 186, and a system control function 187.

The B-mode processing function 181 is a function of generating B-mode data based on a reception signal received from the ultrasonic reception circuit 120. In the B-mode processing function 181, the processing circuit 180 performs, for example, envelope detection processing, logarithmic processing, and the like for reception signals received from the ultrasonic reception circuit 120 to generate data (B-mode data) whose signal intensity is expressed by a luminance level. The generated B-mode data is stored as B-mode raw data on two-dimensional ultrasonic scanning lines (raster) in a raw data memory (not shown).

The Doppler processing function 182 is a function of generating data (Doppler information) by extracting motion information based on the Doppler effect of a moving object in an ROI (Region Of Interest) set in a scan region by performing frequency analysis of reception signals received from the ultrasonic reception circuit 120. The generated Doppler information is stored as Doppler raw data (to be also referred to as Doppler data) on two-dimensional ultrasonic scanning lines in the raw data memory (not shown).

More specifically, the processing circuit 180 causes the Doppler processing function 182 to estimate, for example, as the motion information of the moving object, an average velocity, a mean variance value, an average power value, and the like at each of a plurality of sampling points and generate Doppler data representing the estimated motion information. The moving body is, for example, a blood flow, the tissue such as the cardia wall, or a contrast medium. The processing circuit 180 causes the Doppler processing function 182 to estimate, as the motion information (blood flow information) of a blood flow, the average velocity of the blood flow, the variance value of blood flow velocities, the power value of a blood flow signal at each of a plurality of sampling points and generate Doppler data representing the estimated blood flow information.

The processing circuit 180 causes the Doppler processing function 182 to multiply, for example, the reception signal received from the ultrasonic reception circuit 120 by a reference frequency that is a frequency as a reference for the detection of a Doppler shift by quadrature detection processing and apply a low pass filter (LPF) to the resultant signal, thereby extracting the Doppler shift. Thereafter, the processing circuit 180 causes the Doppler processing function 182 to extract a signal by gating a signal corresponding to a region to be observed and apply a wall filer to the extracted signal. The processing circuit 180 may cause the Doppler processing function 182 to generate Doppler data by applying A/D conversion processing and FFT processing to the signal obtained by the application of the wall filter.

In addition, the processing circuit 180 can cause the Doppler processing function 182 to execute a color Doppler method also called the CFM (Color Flow Mapping) method. The CFM method is configured to perform transmission/reception of ultrasonic waves on a plurality of scanning lines. In the CFM method, for example, an MTI (Moving Target Indicator) filter is applied to a data string at the same position to suppress signals (clutter signals) originating from the stationary tissue or slow-moving tissue and extract a signal originating from a blood flow. The CFM method is configured to estimate blood flow information such as a blood flow velocity, a blood flow variance, and a blood flow power by using the extracted blood flow signal. The image generating function 183 (to be described later) generates the distribution of the estimated blood flow information as, for example, ultrasonic image data displayed two-dimensionally in color (colored Doppler image data). Assume that color display is configured to display the distribution of blood flow information in association with predetermined color codes and also includes gray scales.

Blood flow imaging modes include various types depending on desired clinical information. The modes used in general are a blood flow imaging mode for velocity display that allows the visualization of a blood flow direction and a blood flow average velocity and a blood flow imaging mode for power display that allows the visualization of the power of a blood flow signal.

The velocity imaging mode for velocity display is a mode of displaying colors corresponding to Doppler shift frequencies depending on a blood flow direction and a blood flow average velocity. For example, in the blood flow imaging mode for velocity display, a blood flow in an approaching direction is expressed by a reddish color, and a blood flow in a separating direction is expressed by a bluish color, while a difference in velocity is represented by a difference in hue. The blood flow imaging mode for velocity display is sometimes called a color Doppler mode or color Doppler imaging (CDI) mode.

The blood flow imaging mode for power display is a mode of expressing the power of a blood flow signal by a reddish color hue, color lightness (brightness), or change in color saturation. The blood flow imaging mode for power display is sometimes called the power Doppler (PD) mode. The blood flow imaging mode for power display can draw a blood flow with higher sensitivity than the blood flow imaging mode for velocity display and hence may also be called a high-sensitivity blood flow imaging mode.

The image generating function 183 is a function of generating B-mode image data based on the data generated by the B-mode processing function 181. For example, in the image generating function 183, the processing circuit 180 generates image data for display (display image data) by converting (scan-converting) a scanning line signal sequence of ultrasonic scanning into a scanning line signal sequence in a video format represented by TV or the like. More specifically, the processing circuit 180 generates two-dimensional B-mode image data (to be also referred to as ultrasonic image data) constituted by pixels by executing raw-pixel conversion for B-mode raw data stored in the raw data memory, for example, coordinate conversion corresponding to the ultrasonic scanning form employed by the ultrasonic probe 101. In other words, the processing circuit 180 causes the image generating function 183 to generate a plurality of ultrasonic images (medical images) respectively corresponding to a plurality of consecutive frames by the transmission/reception of ultrasonic waves.

In addition, the image generating function 183 also has a function of generating Doppler waveform data and Doppler image data based on the data generated by the Doppler processing function 182. For example, the image generating function 183 generates Doppler image data with blood flow information being videoed by executing raw-pixel conversion with respect to Doppler raw data stored in the raw data memory. Doppler image data is average velocity image data, variance image data, power image data, or image data obtained by combining them. The processing circuit 180 generates, as Doppler image data, color Doppler image data representing blood flow information in color and Doppler image data representing one piece of blood flow information in a wave shape based on grayscale. Color Doppler image data is generated at the time of executing the above blood flow video mode.

In addition, the image generating function 183 generates a contrast-enhanced image based on a contrast-enhanced imaging signal (to be described later).

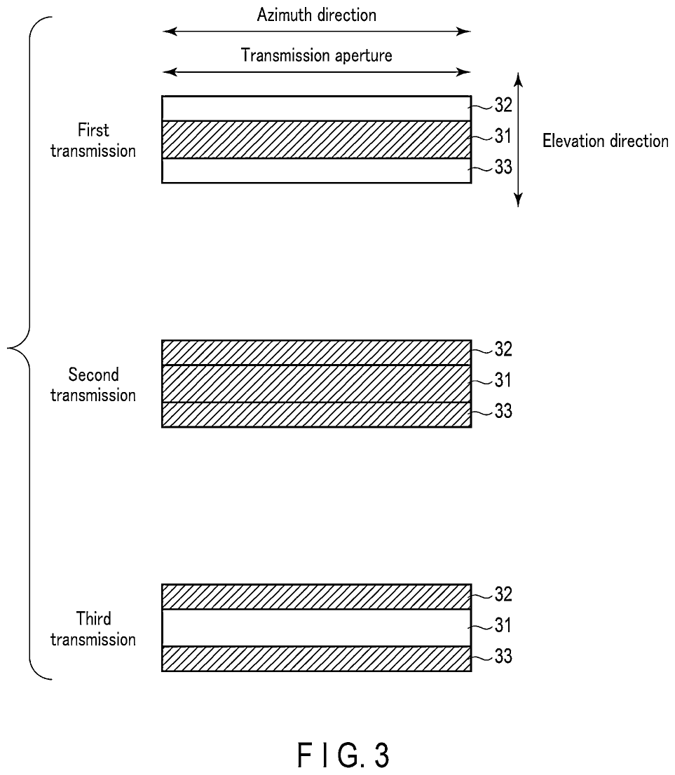

The aperture control function 184 controls the transmission aperture of the ultrasonic probe for each transmission aperture of the ultrasonic probe divided in the elevation direction so as to match an area and a position of elements which form one of ultrasonic beams transmitted a plurality of times with an area and a position of elements which form the remaining ultrasonic beams. The transmission aperture control scheme adopted by the aperture control function 184 will be described later with reference to FIG. 3 and subsequent drawings.

The determination function 185 determines whether bioacoustic shadow has occurred in a contrast-enhanced image.

The display control function 186 is a function of causing the display as the output device 103 to display an image based on each of various types of ultrasonic image data generated by the image generating function 183. More specifically, for example, the processing circuit 180 causes the display control function 186 to perform display control on display of images based on B-mode image data, Doppler waveform data, Doppler image data, generated by the image generating function 183, or image data including all of them.

More specifically, the processing circuit 180 causes the display control function 186 to generate display image data by converting (scan-converting) a scanning line signal sequence of ultrasonic scanning into a scanning line signal sequence in a video format represented by TV or the like. In addition, the processing circuit 180 may execute various types of processing associated with a dynamic range, luminance (brightness), contrast, y curve correction, RGB conversion, and the like. The processing circuit 180 may also add supplementary information such as character information of various types of parameters, scale marks, and body marks to display image data. Furthermore, the processing circuit 180 may generate a GUI (Graphic User Interface) for allowing the operator to input various types of instructions with the input device and cause the display to display the GUI.

The system control function 187 is a function of comprehensively controlling the overall operation of the ultrasonic diagnostic apparatus 1.

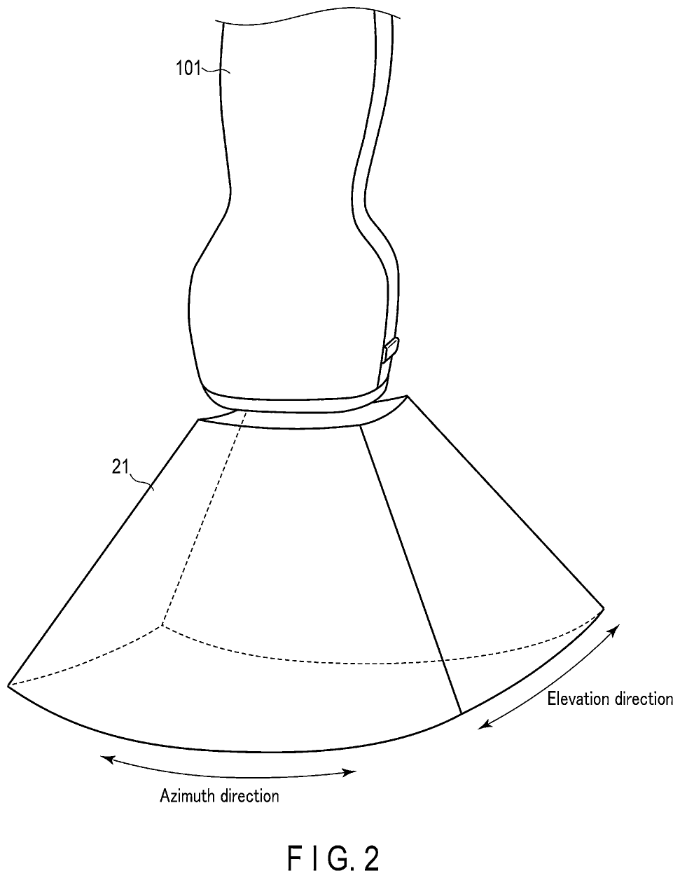

The definition of an ultrasonic beam direction according to this embodiment will be described next with reference to the schematic view of FIG. 2.

FIG. 2 is a schematic view of an ultrasonic beam 21 transmitted from the acoustic radiation surface of the ultrasonic probe 101. This embodiment assumes a 1.5-dimensional array probe or 2-dimensional array probe having elements arranged in a lattice pattern. The scanning direction of an ultrasonic beam transmitted from the ultrasonic probe 101 may be called an azimuth direction. A direction perpendicular to the radiation direction (beam direction) of the ultrasonic beam 21 is an elevation direction and is also called a slice direction.

The first example of the transmission aperture control scheme according to the first embodiment will be described next with reference to FIG. 3.

FIG. 3 is a view when the element array of the ultrasonic probe 101 is seen from the beam direction. Elements used as the transmission aperture is indicated by oblique lines. In this case, the horizontal direction is the azimuth direction, and the vertical direction is the elevation direction. Hereinafter, a similar illustration format is indicated relating to FIGS of the transmission aperture control scheme. In addition, in contrast-enhanced imaging, ultrasonic beams transmitted three times are assumed as one signal group. That is, a contrast-enhanced image is generated based on three echo signals corresponding to ultrasonic beams transmitted three times. Note that a signal group is not necessarily defined by three transmissions of ultrasonic beams and may be defined by three or more transmissions of ultrasonic beams. Note that as the number of times of transmissions of ultrasonic beams in one signal group increases, the time required to generate one contrast-enhanced image increases. For this reason, the number of times of transmissions of ultrasonic beams included in a signal group may be determined appropriately in accordance with the permitted update rate of a contrast-enhanced image.

The case shown in FIG. 3 assumes three element rows divided in the elevation direction. The area of an element row 31 located at a middle portion is larger than the area of an element row 32 and an element row 33 respectively located at end portions. Assume also that three transmissions of an ultrasonic beam in the AM method correspond to one signal group for the generation of a contrast-enhanced image. Note that in the following drawings, each transmission aperture for the transmission of an ultrasonic beam is expressed by hatching. In addition, for the sake of descriptive convenience, the illustration of the delimiters between the elements in the azimuth direction, that is, the formation position of one channel, will be omitted.

In the first transmission that is the transmission performed first for the formation of a signal group, a region corresponding to the element area of the element row 31 located at a middle portion of the element array in the elevation direction is set as a transmission aperture, and the first ultrasonic beam is transmitted through the transmission aperture.

In the second transmission that is the transmission performed second for the formation of a signal group, a region corresponding to the area of all the elements is set as a transmission aperture, and the second ultrasonic beam is transmitted through the transmission aperture.

In the third transmission that is the transmission performed third for the formation of a signal group, regions corresponding to the areas of the two element rows 32 and 33 located at end portions of the element array in the elevation direction are set as transmission apertures, and the third ultrasonic beam is transmitted through the transmission apertures. In this case, the second ultrasonic beam is also called some ultrasonic beams, and the first and third ultrasonic beams are also called the remaining ultrasonic beams.

As described above, the processing circuit 180 causes the aperture control function 184 to control a transmission aperture for each transmission so as to match the size of ultrasonic beams associated with the first transmission and the third transmission with the size of an ultrasonic beam through all the elements associated with the second transmission.

Note that the order of the positions and sizes of the transmission apertures in the first transmission to the third transmission is not limited to the case in FIG. 3 and ultrasonic beams may be transmitted in any order. For example, the transmission of an ultrasonic beam through the transmission aperture corresponding to the area of all the elements in the second transmission shown in FIG. 3 may be the first transmission or the third transmission.

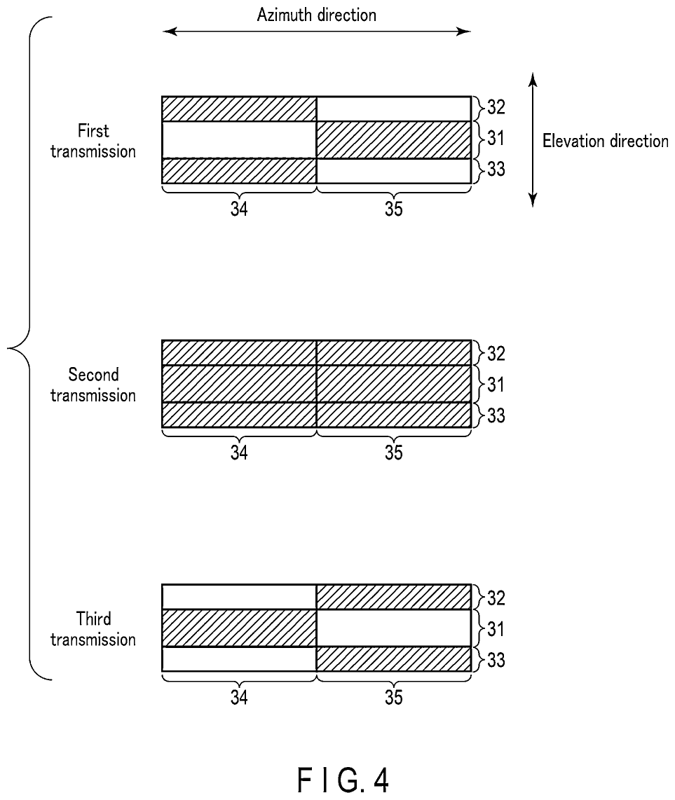

The second example of the transmission aperture control scheme according to the first embodiment will be described next with reference to FIG. 4.

In the case in FIG. 4, the transmission aperture of one element row is divided into two parts in the azimuth direction unlike in the case in FIG. 3 where the one whole element row is set as a transmission aperture.

In the first transmission that is the transmission performed first, a left half region 34 of the element row 32, a right half region 35 of the element row 31, and a left half region 34 of the element row 33 are set as transmission apertures, and the first ultrasonic beam is transmitted through the transmission apertures. That is, the transmission apertures of the respective element rows are alternately set.

In the second transmission that is the transmission performed second, a region corresponding to the area of all the elements is set as a transmission aperture, and the second ultrasonic beam is transmitted through the transmission aperture.

In the third transmission that is the transmission performed third, the right half region 35 of the element row 32, the left half region 34 of the element row 31, and the right half region 35 of the element row 33 are set as transmission apertures, and the third ultrasonic beam is transmitted through the transmission apertures. That is, the elements that have not been used in the first transmission are set as transmission apertures, and the transmission apertures of the respective element rows are alternately set as in the first transmission.

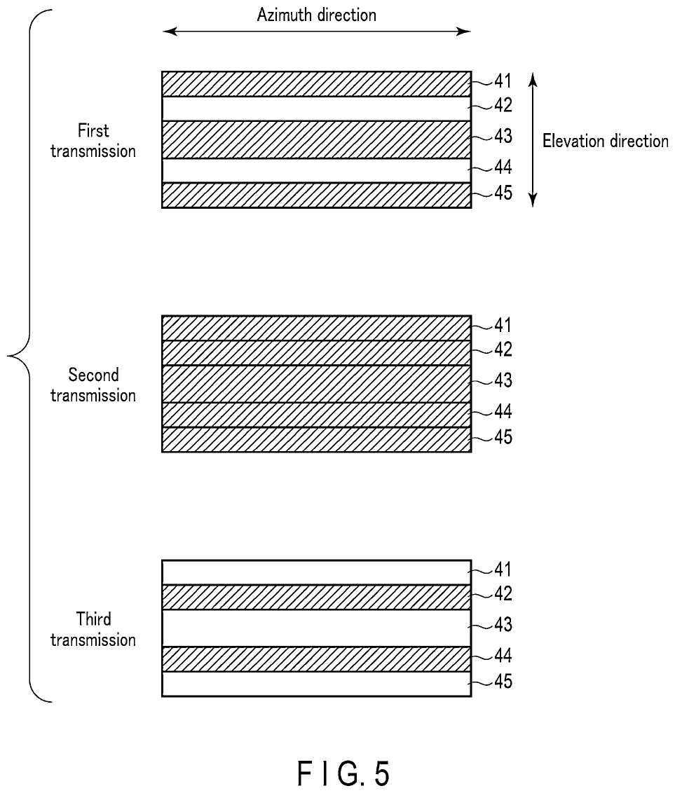

The third example of the transmission aperture control scheme according to the first embodiment will be described next with reference to FIG. 5.

FIG. 5 shows a case where aperture control is performed using five element rows. With regard to five element rows, for example, in the case of a 1.5-dimensional probe, five element rows may be designed by controlling the element rows 32 and 33 at the end portions upon further dividing the element rows. In the case of a 2-dimensional array probe, aperture control may be performed so as to set five element rows in the elevation direction.

In the first transmission that is the transmission performed first, regions corresponding to the areas of the odd-numbered (first, third, and fifth) element rows, of the element rows, which are counted from above in the elevation direction, in other words, an element row 41, an element row 43, and an element row 45, which are the element row at the middle portion and the two element rows at the end portions, are set as transmission apertures, and the first ultrasonic beam is transmitted through the transmission apertures.

In the second transmission that is the transmission performed second, a region corresponding to the area of all the elements is set as a transmission aperture, and the second ultrasonic beam is transmitted through the transmission aperture.

In the third transmission that is the transmission performed third, regions corresponding to the areas of the even-numbered (second and fourth) element rows, of the element rows, which are counted from above in the elevation direction, in other words, an element row 42 and an element row 44, which are the two element rows adjacent to the element row 43, are set as transmission apertures, and the third ultrasonic beam is transmitted through the transmission apertures.

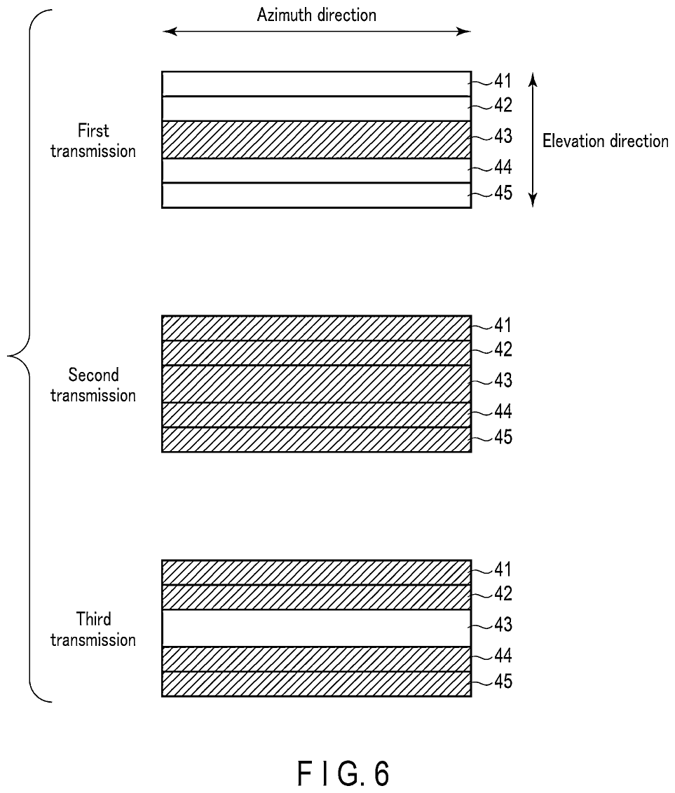

The fourth example of the transmission aperture control scheme according to the first embodiment will be described next with reference to FIG. 6.

FIG. 6 shows a case where the position of a transmission aperture in one transmission is different from that in FIG. 5.

In the first transmission that is the transmission performed first, a region corresponding to the area of the elements of the element row 43 at the middle portion is set as a transmission aperture, and the first ultrasonic beam is transmitted through the transmission aperture.

In the second transmission that is the transmission performed second, a region corresponding to the area of all the elements is set as a transmission aperture, and the second ultrasonic beam is transmitted through the transmission aperture.

In the third transmission that is the transmission performed third, regions corresponding to the area of a plurality of element rows, of the element rows, other than the element row 43 at the middle portion, that is, regions corresponding to the areas of the elements of the element row 41, the element row 42, the element row 44, and the element row 45 are set as transmission apertures, and the third ultrasonic beam is transmitted through the transmission apertures.

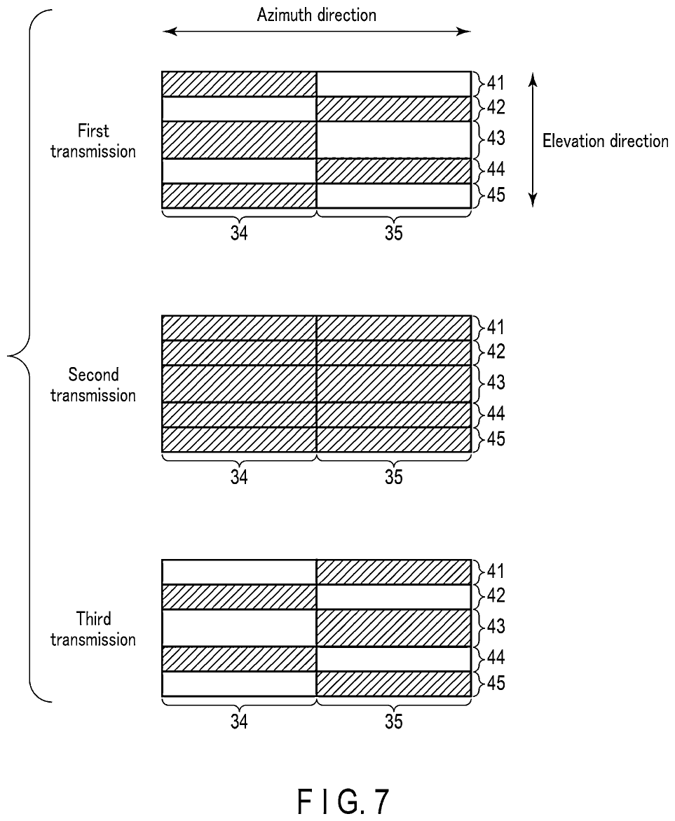

The fifth example of the transmission aperture control scheme according to the first embodiment will be described next with reference to FIG. 7.

FIG. 7 shows a case where each of the five element rows shown in FIGS. 5 and 6 is divided into two transmission apertures in the azimuth direction.

In the first transmission that is the transmission performed first, the left half region 34 of the element row 41, the right half region 35 of the element row 42, the left half region 34 of the element row 43, the right half region 35 of the element row 44, and the left half region 34 of the element row 45 are set as transmission apertures, and the first ultrasonic beam is transmitted through the transmission apertures. That is, the transmission apertures of the respective element rows are alternately set.

In the second transmission that is the transmission performed second, a region corresponding to the area of all the elements is set as a transmission aperture, and the second ultrasonic beam is transmitted through the transmission aperture.

In the third transmission that is the transmission performed third, the right half region 35 of the element row 41, the left half region 34 of the element row 42, the right half region 35 of the element row 43, the left half region 34 of the element row 44, and the right half region 35 of the element row 45 are set as transmission apertures, and the third ultrasonic beam is transmitted through the transmission apertures. That is, the elements that have not been used in the first transmission are set as transmission apertures, and the transmission apertures of the respective element rows are alternately set as in the first transmission.

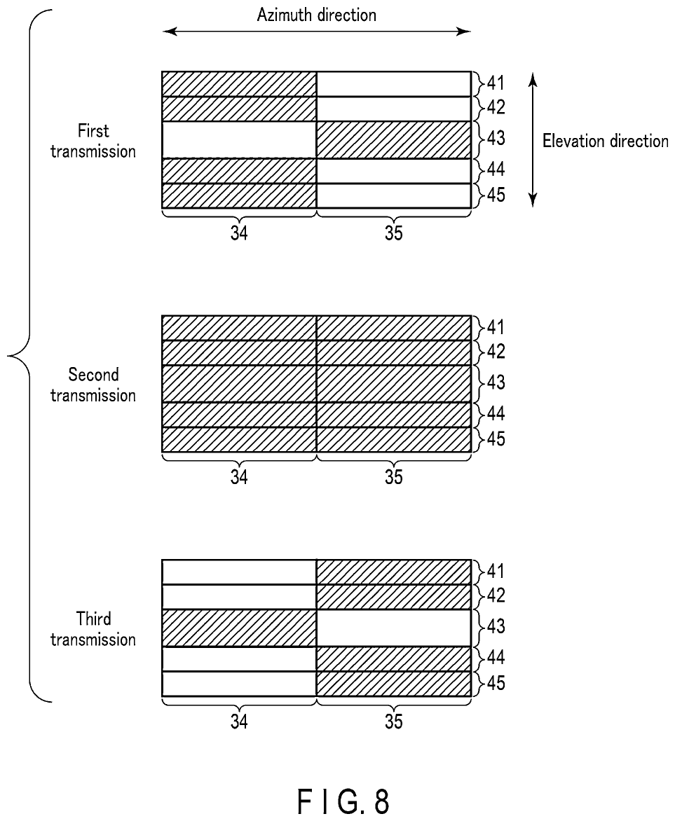

The sixth example of the transmission aperture control scheme according to the first embodiment will be described next with reference to FIG. 8.

FIG. 8 shows a case where the positions of the transmission apertures in one transmission are different from those in FIG. 7.

In the first transmission that is the transmission performed first, the left half region 34 of the element row 41, the left half region 34 of the element row 42, the right half region 35 of the element row 43, the left half region 34 of the element row 44, and the left half region 34 of the element row 45 are set as transmission apertures, and the first ultrasonic beam is transmitted through the transmission apertures.

In the second transmission that is the transmission performed second, a region corresponding to the area of all the elements is set as a transmission aperture, and the second ultrasonic beam is transmitted through the transmission aperture.

In the third transmission that is the transmission performed third, the right half region 35 of the element row 41, the right half region 35 of the element row 42, the left half region 34 of the element row 43, the right half region 35 of the element row 44, and the right half region 35 of the element row 45 are set as transmission apertures, and the third ultrasonic beam is transmitted through the transmission apertures. That is, the elements that have not been used in the first transmission are set as transmission apertures, and the transmission apertures of the respective element rows are alternately set as in the first transmission.

Note that the positions and the sizes of the transmission apertures are not limited to those in the examples shown in FIGS. 3, 4, 5, 6, 7, and 8. In transmitting ultrasonic beams other than the ultrasonic beam in the second transmission, that is, a plurality of ultrasonic beams in cases other than the case of transmitting an ultrasonic beam from all the elements, the processing circuit 180 may cause the aperture control function 184 to perform aperture control so as to set the size of a transmission aperture corresponding to the element width in the azimuth direction for each ultrasonic beam and so as not to transmit ultrasonic beams from some of the element rows adjacent to each other in the elevation direction. In other words, for the remaining ultrasonic beams in the signal group, aperture control may be performed to make the respective ultrasonic beams have different patterns.

With this control, each transmission aperture is divided into two parts in the azimuth direction while the width of each transmission aperture in the azimuth direction is maintained unlike the sparce division scheme of setting a transmission aperture for each channel. It is therefore possible to reduce the influence of crosstalk.

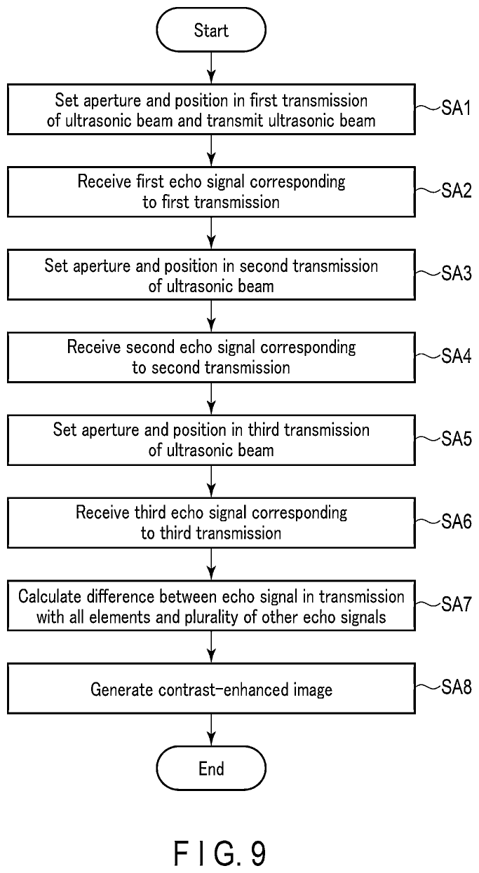

Contrast-enhanced imaging processing by the ultrasonic diagnostic apparatus 1 according to the first embodiment will be described next with reference to the flowchart of FIG. 9.

In step SA1, the processing circuit 180 causes the aperture control function 184 to set the position and the size of each transmission aperture associated with the first transmission according to the transmission aperture control schemes shown in FIGS. 3, 4, 5, 6, 7, and 8 described above. Subsequently, the ultrasonic transmission circuit 110 transmits the first ultrasonic beam.

In step SA2, for example, the ultrasonic reception circuit 120 receives the first echo signal as the reflected wave of the first ultrasonic beam from the living body P.

In step SA3, the processing circuit 180 causes the aperture control function 184 to set the position and the size of each transmission aperture associated with the second transmission. Subsequently, the ultrasonic transmission circuit 110 transmits the second ultrasonic beam.

In step SA4, for example, the ultrasonic reception circuit 120 receives the first echo signal as the reflected wave of the second ultrasonic beam from the living body P.

In step SA5, the processing circuit 180 causes the aperture control function 184 to set the position and the size of each transmission aperture associated with the third transmission. Subsequently, the ultrasonic transmission circuit 110 transmits the third ultrasonic beam.

In step SA6, for example, the ultrasonic reception circuit 120 receives the third echo signal as the reflected wave of the third ultrasonic beam from the living body P.

In step SA7, for example, the image generating function 183 or the system control function 187 generates a differential signal (contrast-enhanced imaging signal) by calculating the difference between some echo signals, of a plurality of echo signals corresponding to a signal group, which correspond to an ultrasonic beam in transmission with all elements and a plurality of echo signals corresponding to a plurality of remaining ultrasonic beams and calculates a differential signal (contrast-enhanced imaging signal). For example, in the examples shown in FIGS. 3, 4, 5, 6, 7, and 8, the difference between an echo signal r_{TxF} (t) corresponding to the second transmission that is the transmission of an ultrasonic beam in transmission with all elements and the sum of an echo signal r_{TxP1} (t) corresponding to the first transmission and an echo signal F_{TxP2} (t) corresponding to the third transmission is set as a contrast-enhanced imaging signal ram (t). That is, the contrast-enhanced imaging signal ram (t) is calculated according to equation (1).

$${r}_{AM} ( t ) = {r}_{T x P 1} ( t ) + {r}_{T x P 2} ( t ) - {r}_{T x F} ( t ) \qquad ( 1 )$$

Although this case assumes the processing of calculating a difference with respect to an echo signal on the reception side, the transmission side may perform control to facilitate the calculation of a differential signal. If, for example, the transmission of an ultrasonic beam with all the element being set as a transmission aperture corresponds to the middle in transmission processing of signal groups, that is, the second transmission in the examples in FIGS. 3, 4, 5, 6, 7, and 8, the processing circuit 180 may cause the aperture control function 184 to invert the polarity of each ultrasonic beam for each transmission. This allows the reception side to generate a contrast-enhanced imaging signal by only applying addition processing to an echo signal as indicated by equation (1). Alternatively, the phase of each ultrasonic beam may be inverted for each transmission to make it possible to generate a contrast-enhanced imaging signal by only applying addition processing to an echo signal on the reception side in the same manner as described above.

In step SA8, the processing circuit 180 causes the image generating function 183 to generate a contrast-enhanced image from the contrast-enhanced imaging signal ram (t).

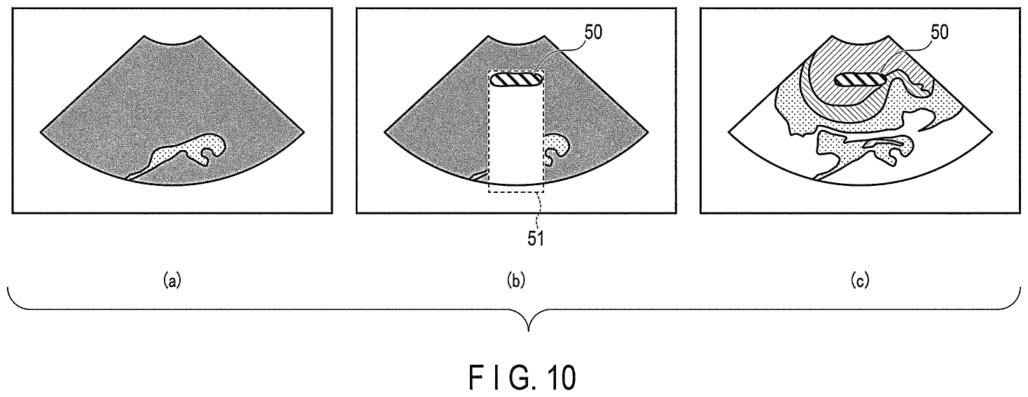

A contrast-enhanced image collected by the transmission aperture control scheme according to the first embodiment will be described next with reference to the conceptual views of FIGS. 10 (a), 10 (b), and 10 (c).

FIG. 10 (a) is a schematic view of a contrast-enhanced image generated by the transmission aperture control scheme according to the first embodiment. FIG. 10 (b) is a schematic view of a contrast-enhanced image generated by the transmission aperture division scheme as a prior art. FIG. 10 (c) is a schematic view of a contrast-enhanced image generated by the sparce transmission aperture scheme as a prior art. Assume a case where the three contrast-enhanced images were obtained by imaging an imaging region of the same imaging target.

The contrast-enhanced image according to the prior art indicated in FIG. 10 (c) exhibits relatively poor tissue cancellation performance because of the large influence of crosstalk from transmitting channels to non-transmitting channels. Accordingly, the tissue is depicted unlike an ideal contrast-enhanced image having no tissue depicted.

The contrast-enhanced image according to the prior art indicated in FIG. 10 (b) exhibits higher tissue cancellation performance than the contrast-enhanced image indicated in FIG. 10 (c) because of the small influence of crosstalk. The scheme for the contrast-enhanced image indicated in FIG. 10 (b) is smaller in transmission aperture in the azimuth direction than the sparce transmission aperture scheme as the prior art and hence cannot receive an echo signal from a position deeper than a tumor 50. Consequently, this image includes a bioacoustic shadow 51. Therefore, this scheme may not acquire necessary contrast-enhanced information at a given depth or more.

The contrast-enhanced image indicated in FIG. 10 (a), which is acquired by the transmission aperture control scheme according to the first embodiment, exhibits less influence of crosstalk from transmitting channels to non-transmitting channels because transmission apertures are designed with each transmission aperture being divided into two parts. This can improve the tissue cancellation performance. In addition, in one transmission, since a transmission aperture throughout the whole elements is maintained in the azimuth direction, the influence of a bioacoustic shadow can be reduced. Accordingly, as compared with the contrast-enhanced images indicated in FIGS. 10 (b) and 10 (c) as the prior arts, the contrast-enhanced image indicated in FIG. 10 (a) has a good image quality.

Note that the transmission aperture control schemes may be switched depending on whether a bioacoustic shadow has occurred.

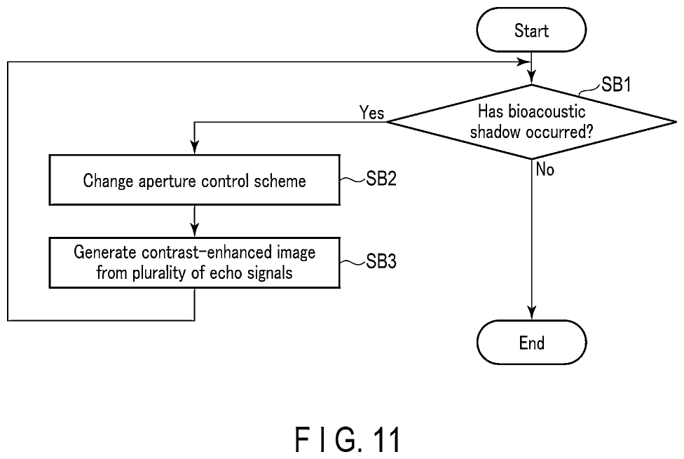

Transmission aperture control scheme switching processing will be described with reference to the flowchart of FIG. 11.

In step SB1, the processing circuit 180 causes the determination function 185 to determine whether a bioacoustic shadow has occurred in the contrast-enhanced image. More specifically, assume that a region with pixel values equal to or less than a first threshold is extracted, and the extracted region has a rectangular shape at a given depth and more. In this case, if the difference from a pixel value at a pixel position other than those in the rectangular region at the given depth and more is equal to or more than a second threshold, the determination function 185 may determine that a bioacoustic shadow has occurred. Alternatively, pattern recognition processing may be performed as follows. A pattern shape unique to a bioacoustic shadow is registered. Pattern matching is then performed to determine whether a pattern shape matching the bioacoustic shadow is present on the contrast-enhanced image, thereby determining the occurrence of a bioacoustic shadow. Alternatively, a contrast-enhanced image on which a bioacoustic shadow has occurred is used as input data using a machine learning model, and the machine learning model represented by a neural network is trained using a contrast-enhanced image with an annotation being added to the region of the bioacoustic shadow by, for example, a manual operation, thereby generating a learned model. The occurrence of a bioacoustic shadow in the contrast-enhanced image may be determined by using the learned model.

If a bioacoustic shadow has occurred, the process advances to step SB2. If no bioacoustic shadow has occurred, the processing is terminated.

In step SB2, the processing circuit 180 causes the aperture control function 184 to change the transmission aperture control scheme for the ultrasonic probe in use to another transmission aperture control scheme. For example, if the transmission aperture control scheme shown in FIG. 4 is executed, the aperture control function 184 may perform the processing of changing the scheme to the transmission aperture control scheme shown in FIG. 3.

In step SB3, the processing circuit 180 causes the image generating function 183 to generate a contrast-enhanced image by using a plurality of echo signals corresponding to a signal group originating from ultrasonic beams transmitted by the new transmission aperture control scheme. The process then returns to step SB1 to repeat similar processing.

According to the first embodiment described above, ultrasonic beams associated with three or more transmissions from the ultrasonic probe are set as one signal group, and the transmission apertures for the ultrasonic beams of the signal group are controlled to match the position and the size of the first transmission aperture corresponding to the first ultrasonic beam of the signal group with the position and the sum of the sizes of transmission apertures for the remaining ultrasonic beams of the signal group.

This will maintain the transmission apertures in the azimuth direction and suppress variation in tissue cancellation performance, thereby maintaining the tissue cancellation performed. In addition, it is possible to reduce the influences of crosstalk and a bioacoustic shadow. That is, it is possible to improve the contrast-enhanced imaging performance.

### Second Embodiment

The first embodiment assumes aperture control designed to transmit ultrasonic beams from all the elements or half the elements in the azimuth direction. The second embodiment differs from the first embodiment in that sparce aperture control is performed with an element group of a plurality of elements in the azimuth direction being a unit of transmission.

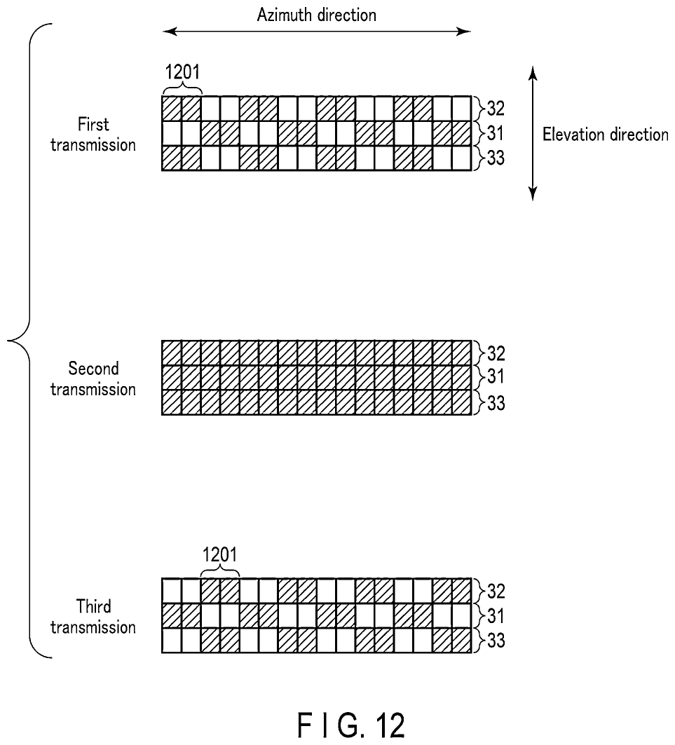

The first example of a transmission aperture control scheme according to the second embodiment will be described with reference to FIG. 12.

Like FIG. 3, FIG. 12 is a view when the element rows are seen from the beam direction. For the sake of descriptive convenience, FIG. 12 shows an example in which 16 elements are arranged in the azimuth direction to explain one delimiter in the azimuth direction as one element. In practice, a 1.5-dimensional or 2-dimensional array ultrasonic probe has several ten or several hundred elements arrayed in the azimuth direction, and the transmission aperture control scheme described below may be applied to each element.

In the first transmission of an ultrasonic beam, a plurality of elements adjacent to each other in the azimuth direction are set as an element group for the transmission of an ultrasonic beam in each element row 31-33. That is, in the example in FIG. 12, two elements adjacent to each other in the azimuth direction are set as an element group 1201. In addition, the element groups 1201 adjacent to each other in the azimuth direction are arranged at two-element intervals instead of being consecutively arranged.

In addition, the respective element groups 1201 are arranged so as not to be adjacent to each other in the elevation direction. In other words, the element groups 1201 are arranged in a checker pattern in the elevation direction.

In the second transmission of an ultrasonic beam, as in the first embodiment, a region corresponding to the area of all the elements is set as a transmission aperture, and the second ultrasonic beam is transmitted through the transmission aperture.

In the third transmission of an ultrasonic beam, the elements that have not been used for the first transmission of an ultrasonic beam but are targeted at the first transmission are set as the element groups 1201, and an ultrasonic beam is transmitted.

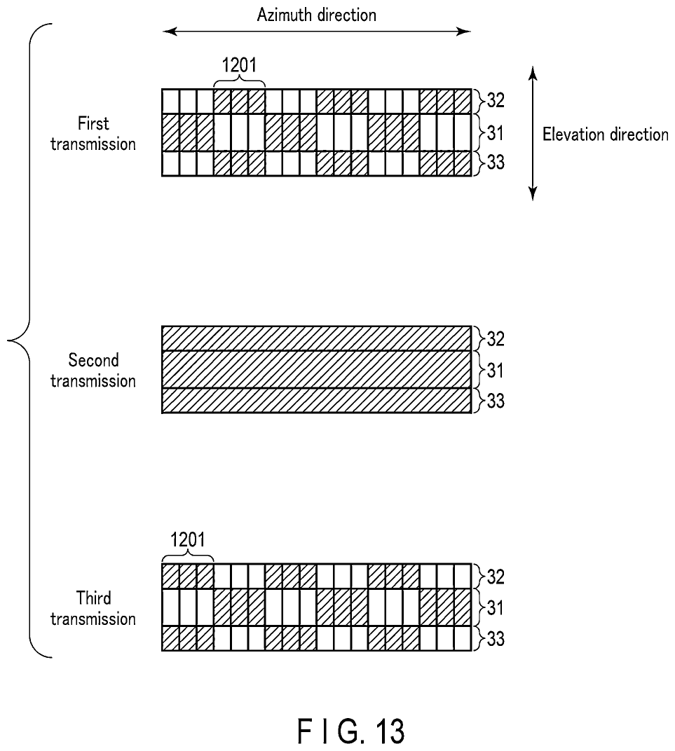

The second example of the transmission aperture control scheme according to the second embodiment will be described next with reference to FIG. 13.

The second example of the second embodiment is an example in which three elements are set as one element group 1201. As in the first example of the second embodiment, in this case, the element groups 1201 adjacent to each other in the azimuth direction are arranged at intervals of three elements equivalent to each element group. In addition, the respective element groups 1201 are arranged in a checker pattern in the elevation direction so as not to be adjacent to each other in a direction in which the element rows are orthogonal to each other.

Note that the interval between the element groups 1201 adjacent to each other in the azimuth direction is not limited to that equivalent to the number of elements constituting the element group 1201 and may be an interval equivalent to two or more elements. If, for example, five elements are set as one element group 1201, the interval between the adjacent element groups 1201 may be equivalent to two elements. That is, the interval between the adjacent element groups 1201 may be equivalent to at least two elements. Assume that in the first transmission of an ultrasonic beam, an ultrasonic beam is transmitted through the element group 1201 including five elements. In this case, in the third transmission, two elements corresponding to the interval between the element groups 1201 in the first transmission may be set as the element group 1201, and an ultrasonic beam may be transmitted from the element group 1201. That is, even if the first transmission of an ultrasonic beam differs from the third transmission of an ultrasonic beam in the number of elements constituting the element group 1201, the first transmission and the third transmission may have a complementary relationship such that transmission in the first transmission and the third transmission can be regarded as transmission from all the elements.

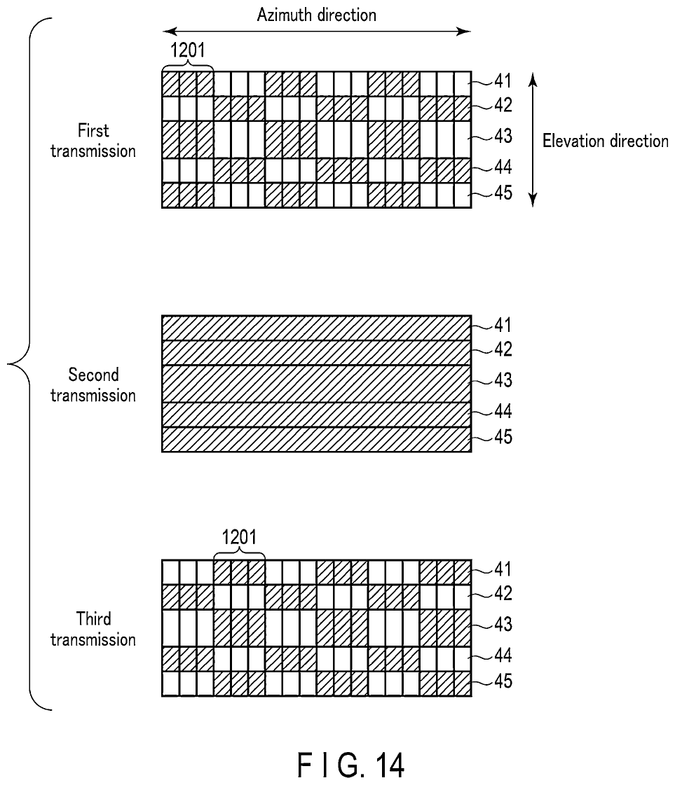

The third example of the transmission aperture control scheme according to the second embodiment will be described next with reference to FIG. 14.

The third example of the second embodiment indicates a case where five element rows are present in the elevation direction. In the example in FIG. 14, three elements adjacent to each other in the azimuth direction are set as one element group 1201, and the respective element groups 1201 are arranged in a checker pattern in the elevation direction so as not to be adjacent to each other. As described above, even if the number of element rows in the elevation direction is three or more, the element group 1201 can be set in the same manner as described above.

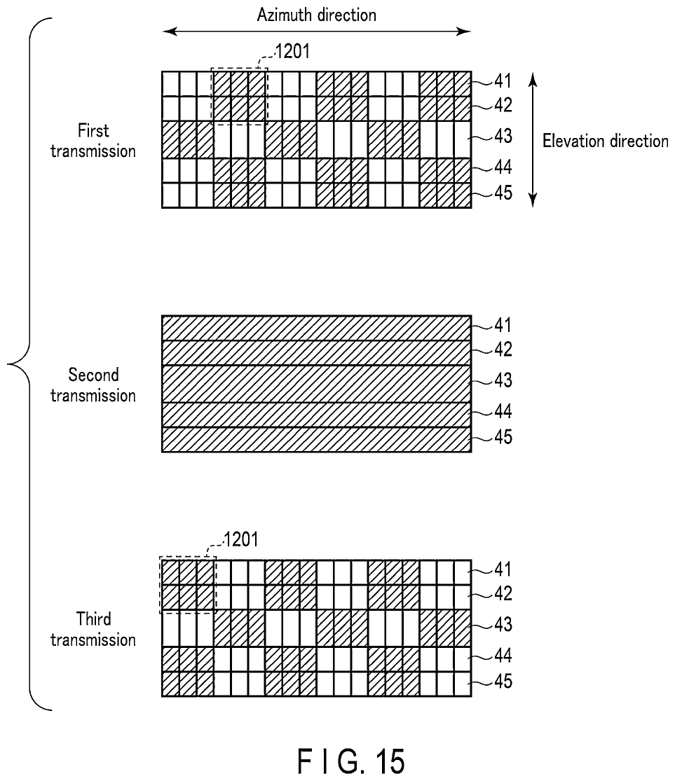

The fourth example of the transmission aperture control scheme according to the second embodiment will be described next with reference to FIG. 15.

Like the third example, the fourth example according to the second embodiment indicates a case where five element rows are present in the elevation direction. In this case, however, three elements adjacent to each other in the elevation direction and two elements adjacent to each other in the elevation direction are set as one element group 1201. In this manner, the size of an element group in the elevation direction may be changed.

According to the second embodiment described above, an ultrasonic beam is transmitted a plurality of times upon setting of element rows in a checker pattern so as to group a plurality of elements into element groups in the azimuth direction and set the interval between the element groups adjacent to each other in the azimuth direction to two or more elements and so as not to locate the element groups adjacent to each other in the elevation direction. This makes it possible to reduce the influence of crosstalk from transmitting channels to non-transmitting channels by setting the interval between the element groups to two elements, that is, two or more channels. Ultrasonic beams in a general sparce transmission aperture control scheme with an interval of two or more channels cause grating lobe because the virtual element pitch becomes rough. The ultrasonic diagnostic apparatus according to the second embodiment can complement an acoustic field because element positions are alternately arranged sparsely in the elevation direction, that is, in a checker pattern, thereby reducing grating lobe.

Note that the term “processor” used in the above explanation means, for example, a Central Processing Unit (CPU), a Graphics Processing Unit (GPU), or a circuit such as an Application Specific Integrated Circuit (ASIC) or a programmable logic device (for example, a Simple Programmable Logic Device (SPLD), a Complex Programmable Logic Device (CPLD), or a Field Programmable Gate Array (FPGA)). If the processor is, for example, a CPU, the processor implements a function by reading out a program stored in a storage circuit and executing it. On the other hand, if the processor is an ASIC, the function is directly incorporated as a logic circuit in the circuit of the processor, instead of storing the program in the storage circuit. Note that the processor according to this embodiment is not necessarily configured as a single circuit for each processor, and a plurality of independent circuits may be combined to form one processor and implement the function. Furthermore, a plurality of constituent elements in the drawings may be integrated to form one processor and implement the function.

In addition, each function according to this embodiment can also be implemented by installing programs for executing the above processing in a computer such as a workstation and expanding them in a memory. In this case, the programs which can cause the computer to execute the above techniques can be distributed by being stored in storage media such as magnetic disks (hard disks and the like), optical disks (CD-ROMs, DVDs, and the like), and semiconductor memories.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasonic diagnostic apparatus comprising:
an ultrasonic probe having a plurality of elements and configured to transmit an ultrasonic beam a plurality of times, the plurality of elements being divided in an elevation direction; and
processing circuitry configured to control a transmission aperture of the ultrasonic probe so as to match an area and a position of elements which form one of ultrasonic beams transmitted the plurality of times with an area and a position of elements which form remaining ultrasonic beams.

2. The ultrasonic diagnostic apparatus according to claim 1, wherein the processing circuitry controls the transmission aperture so as to have a width of the elements of the ultrasonic probe in an azimuth direction for each ultrasonic beam.

3. The ultrasonic diagnostic apparatus according to claim 1, wherein the processing circuitry controls the transmission aperture so as not to transmit the remaining ultrasonic beams from some of elements adjacent to each other in the elevation direction.

4. The ultrasonic diagnostic apparatus according to claim 1, wherein the ultrasonic probe includes three element rows divided in the elevation direction,
the one of the ultrasonic beams are ultrasonic beams from all elements, and
the remaining ultrasonic beams include an ultrasonic beam from one element row located at a middle portion in the azimuth direction and ultrasonic beams from two element rows located at end portions in the azimuth direction.

5. The ultrasonic diagnostic apparatus according to claim 1, wherein the plurality of transmissions of the ultrasonic beams are three transmissions of an ultrasonic beams,
the one of the ultrasonic beams are ultrasonic beams transmitted second, and
the processing circuitry inverts a phase for each transmission.

6. The ultrasonic diagnostic apparatus according to claim 1, wherein the processing circuitry is further configured to generate a contrast-enhanced image based on a differential signal between an echo signal corresponding to the one of the ultrasonic beams and an echo signal corresponding to the remaining ultrasonic beams.

7. The ultrasonic diagnostic apparatus according to claim 6, wherein the processing circuitry is further configured to:
determine whether or not bioacoustic shadow has occurred in the contrast-enhanced image; and
change a transmission aperture control scheme of the ultrasonic probe if the bioacoustic shadow has occurred in the contrast-enhanced image.

8. The ultrasonic diagnostic apparatus according to claim 1, wherein the plurality of times are not less than three.

9. The ultrasonic diagnostic apparatus according to claim 1, wherein the remaining ultrasonic beams include a plurality of ultrasonic beams, and the respective ultrasonic beams have different patterns.

10. The ultrasonic diagnostic apparatus according to claim 1, wherein the processing circuitry sets the transmission aperture of the ultrasonic beam so as to group a plurality of elements adjacent to each other in an azimuth direction of the ultrasonic probe into element groups for transmission of the ultrasonic beam, set an interval corresponding to at least two elements between the element groups adjacent to each other in the azimuth direction, and set the element groups in a checker pattern in the elevation direction.

11. The ultrasonic diagnostic apparatus according to claim 10, wherein the element group includes a plurality of elements adjacent to each other in the elevation direction.
